(19) United States
(12) Patent Application Publication
(10) Pub. No.: US 2004/0142324 A1
(43) Pub. Date: Jul. 22, 2004

(54) Method for extracting nucleic acids

(76) Inventor: Andreas Bosio, Koln (DE)

Correspondence Address: JACOBSON HOLMAN PLLC, 400 SEVENTH STREET N.W., SUITE 600, WASHINGTON, DC, 20004, US

(21) Appl. No.: 10/239,195

(22) PCT Filed: Mar. 30, 2001
(86) PCT No.: PCT/EP01/03669

(30) Foreign Application Priority Data
Mar. 31, 2000 (DE) 100 16 138.3
Apr. 29, 2000 (EP) 00109297.2

(51) Int. Cl. C12Q 1/68; C07H 21/04; C07K 9/00

(52) U.S. Cl. 435/6; 536/23.1; 530/322

(57) ABSTRACT

A nucleic acid complex with an anchor oligonucleotide, an arbitrary nucleic acid or an arbitrary oligonucleotide and a linker nucleic acid, wherein the linker nucleic acid is partially hybridized with the anchor nucleotide and partially hybridized with the arbitrary nucleic acid or the arbitrary oligonucleotide and the anchor nucleotide is immobilized on a support by its 3′-terminus.

The subject matter of the present invention is a nucleic acid complex with an anchor oligonucleotide, an arbitrary nucleic acid or an arbitrary oligonucleotide and a linker oligonucleotide, a nucleic acid or oligonucleotide hybridization product and a method for the manufacturing and separation of nucleic acids or oligonucleotides after the synthesis thereof using the nucleic acid complex of the invention.

Furthermore, the invention relates to a device enabling the extraction of RNA from homogenized tissue or cells, the purification, transcription into cDNA and re-purification thereof in a combined and automatable one vessel process (one tube reaction).

The analysis of gene expression requires the extraction and purification and labeling of mRNA. Frequently, the mRNA labeling is performed by the step of cDNA synthesis since it enables the insertion of labeled nucleotides. The mRNA isolation method comprises the selective extraction of RNA from the mixture of substances existing in a cell or in tissue (lipids, proteins, saccharides, DNA, RNA, low-molecular substances) and the subsequent mRNA enrichment from the tRNA, rRNA, snRNA and mRNA mixture using oligo(dT) nucleic acids which are bonded to a column (affinity chromatography) or (magnetic) spherical particles (batch method). Subsequently, the mRNA is eluted from the oligo(dT) and subjected to further enzymatic reactions when separated from oligo(dT) or enzymatically reacted when still bonded to oligo(dT) (and a solid phase). The reverse transcriptase reaction for preparing cDNAs is an enzymatic reaction which can also be performed in the presence of oligo(dT) and a solid phase coupled thereto. However, the synthesized cDNA cannot easily be separated from the solid phase since the oligo(dT)s serve as primer and thus, eventually, the cDNA is covalently bonded to the solid phase. Hence, a further use of the cDNA requiring the separation thereof from the solid phase is not possible.

WO-A-98/31838 pertains to a method for the identification of gene expression patterns in mRNA populations. The method is useful in the determination of differential gene expressions in different cells or tissues including cells or tissues of target organisms. Methods for the determination of the gene expression frequency in mRNA populations are disclosed whereby a method for the comparison of the gene expression frequency of different cells or tissues becomes available. Also described is a method for the isolation of genes which had been identified by so-called tag sequences according to the method described there. The sequences may be used for the diagnosis of certain diseases.

WO-A-95/13368 pertains to the isolation of a nucleic acid from a sample. For this, the sample is boiled and subsequently cooled. The nucleic acids are precipitated on a solid support. The method is used in the preparation of nucleic acids for a subsequent amplification. In particular, the method can be used in the isolation of nucleic acids of aged, fixed or otherwise treated samples.

WO-A-97/10363 pertains to the serial analysis of Gene Expression (SAGE), a method for the rapid quantitative and qualitative analysis of transcripts. Short, defined sequences corresponding to expressed genes were isolated and analyzed. Sequencing of more than 1,000 defined tags in a short time results in a gene expression pattern characteristic of the function of a cell or a tissue. The SAGE method is useful as an auxiliary method for the identification and isolation of new tag sequences corresponding to novel transcripts and genes.

The technical problem forming the basis of the invention is to provide a simple and reliable method and means for carrying out the method to avoid the above-mentioned drawbacks.

The problem is solved by a nucleic acid complex with an anchor oligonucleotide, an arbitrary nucleic acid or an arbitrary oligonucleotide and a linker oligonucleotide, wherein the linker oligonucleotide is partially hybridized with the anchor oligonucleotide and partially hybridized with the arbitrary nucleic acid or the arbitrary oligonucleotide and the anchor oligonucleotide is immobilized on a support by its 3′-terminus.

The anchor oligonucleotide is immobilized on a support. The linker oligonucleotide has a sequence enabling it to hybridize both with the anchor oligonucleotide and with an additional nucleic acid or an additional oligonucleotide. Hence, the sequence of the linker oligonucleotide must be selected to match with the sequence of the additional nucleic acid/ oligonucleotide. If, e.g., cDNA is to be obtained from mRNA (poly(A⁺)RNA), the linker oligonucleotide has a poly(dT) part.

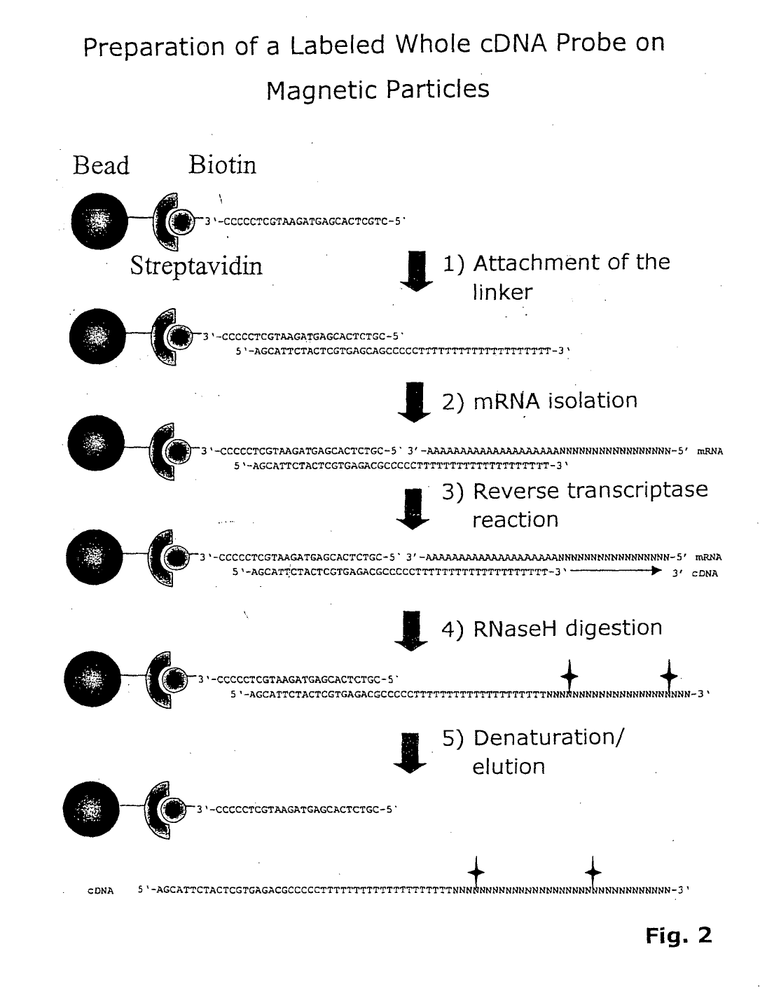

The method of the invention for preparing cDNA is exemplified in more detail in FIG. 2:

A support, preferably a bead, is employed; a linker oligonucleotide is bonded to said support by an anchor oligonucleotide such that a poly(dT) part is present. Now, mRNA is attached to this poly(dT) part, and the linker oligonucleotide is subjected to a common reverse transcriptase reaction—optionally with the insertion of labeled nucleotides—extended by a polymerase reaction. Then, the mRNA can be destroyed by, e.g., RNaseH or NaOH, and the cDNA extended linker oligonucleotide may be split off by increasing the temperature, optionally by changing the buffer.

The method may also be used to synthesize any nucleic acid (having a structure which is known at least partially). For this the linker oligonucleotide. is again required to have a sequence partially overlapping with the target molecule. Then, a nucleic acid complementary to the target nucleic acid—optionally with the insertion of labeled nucleotides—can be synthesized at the linker oligonucleotide. Subsequently, the nucleic acid can be split off and processed as a double strand (having a single strand end). Such a method is useful, e.g., in the direct, non-PCR amplified preparation of cDNA libraries having selectively enriched cDNA molecules corresponding to the selected sequence of the linker oligonucleotide by ligating the produced and isolated double strand cDNAs directly into a suitably prepared cloning vector.

In the nucleic acid complex of the invention, e.g., a temperature from 35° C. to 85° C., preferably from 45° C., to 65° C., results in an anellation of the anchor nucleotide and the linker oligonucleotide.

Preferably, the hybridizing area of the anchor and linker oligonucleotides in the nucleic acid complex of the invention has a range of GC:AT nucleotides from 20:80 to 80:20.

According to the invention, an anellation between the anchor oligonucleotide and the linker oligonucleotide preferably proceeds at a temperature being higher than that of the anellation between the linker oligonucleotide or the arbitrary nucleic acid and the arbitrary oligonucleotide.

In a preferred embodiment the anchore oligonucleotide of the nucleic acid complex is covalently bonded to the support or immobilized thereon by an affinity group.

Another subject of the present invention is a nucleic acid or oligonucleotide hybridization product of an anchor oligonucleotide and a linker oligonucleotide wherein the linker oligonucleotide is partially hybridized with the anchor oligonucleotide and the anchor oligonucleotide is immobilizable on a support by the 3′-terminus.

The anchor oligonucleotide immobilizable on a support by the 3′- terminus and the linker oligonucleotide may be marketed in the form of a kit optionally together with additional components to carry out the method of the invention.

Another subject of the invention is a method for preparing and separating nucleic acids or oligonucleotides after the synthesis thereof using an embodiment of the nucleic acid complex of the invention. To this, the following steps are carried out:

attachment of an arbitrary target nucleic acid, the 5′-terminus of which partially hybridizing with the linker oligonucleotide, wherein the linker oligonucleotide is hybridized with the anchor oligonucleotide immobilized on a support;

extension of the linker oligonucleotide by a polymerase and

separation of the extended linker oligonucleotide optionally together with the target nucleic acid.

Preferably, the target nuclei acid is mRNA. The target nucleic acid can be degraded by RnaseH or NaOH in particular prior to the separation of the extended linker oligonucleotide.

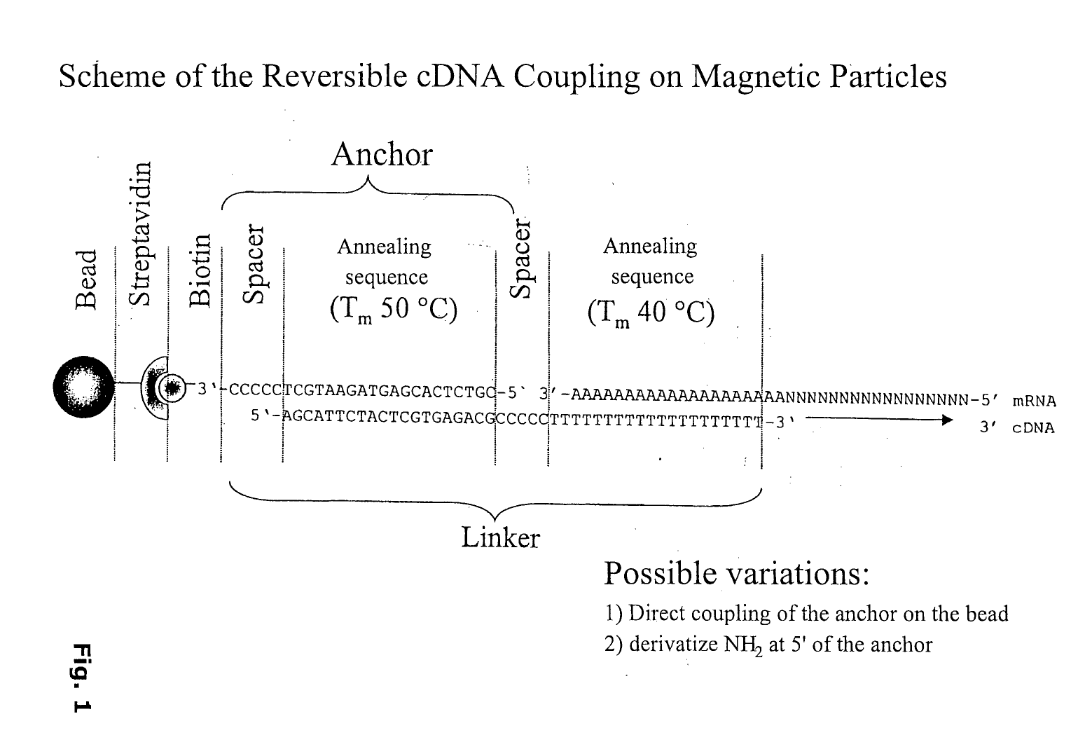

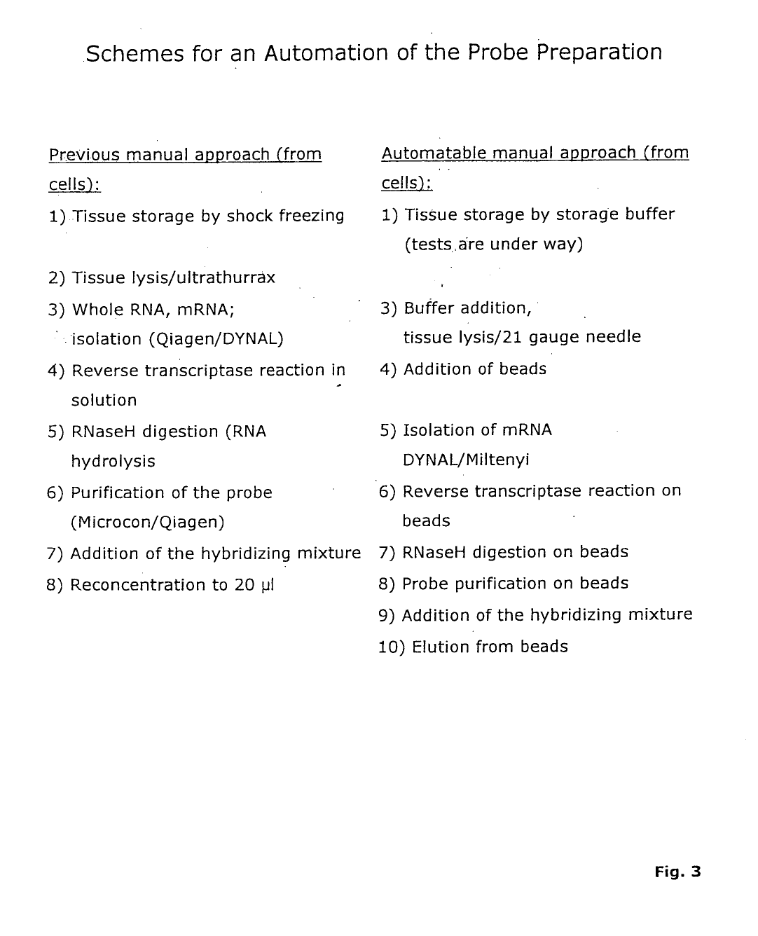

Using the preferred linker and anchor nucleotides illustrated in FIG. 1, the invention enables a temporary non-covalent bonding of cDNA to the solid phase which can be separated by a simple heating step. Therewith all reactions required to isolate, purify, label and re-purify the final probe can be executed on the solid phase (FIG. 2). On the other hand, the execution on the solid phase enables a straightforward automation of the complete sequence (FIG. 3).

The invention will be illustrated in more detail by the following examples. The method of the invention was used to prepare fluorescence label cDNA from whole RNA. Dynabeads M-280 Streptavidin (DYNAL) was used as solid phase. Different quality controls were conducted to examine the mRNA yield, the incorporation efficiency and the purity of the Cy5-labeled cDNA. Subsequently, the labeled cDNA was hybridized together with a cDNA labeled in a conventional manner (Cy3) in a cDNA array. The conventional labeling was performed as follows: insertion of fluorescence labeled Cy3 nucleotides in solution during a reverse transcriptase reaction (superscript II) (GIBCO) starting from purified mRNA which had been isolated from the same whole RNA also used for the other probe, subsequently the separation of mRNY by RnaseH treatment and purification of the labeled cDNA by silica membranes (Qiaquick, QIAGEN). The Cy3Cy5 signal ratio of individual cDNAs immobilized on the array enabled a statement about the performance of the labeling method of the invention compared with conventional labeling methods. To this one proceeded as follows:

a) Preparation of the anchor linker hybrid:

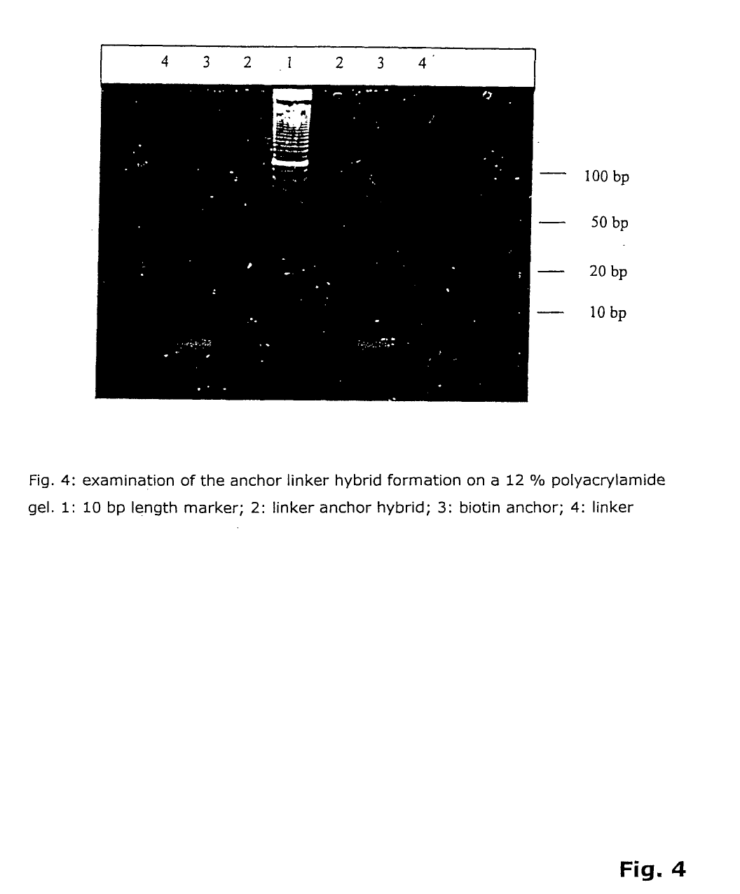

The biotin anchor and the linker were adjusted to concentration of 350 ng/μl in bidistilled water free from RNase. In a 0.2 ml Eppendorf reaction vessel 9.1 μl of biotin anchor (400 pmol; 8000 pg/pmol), 15.6 μl of linker (400 pmol; 13647 pg/pmol) and 25 μl of a solution from 10 mM Tris HCl, pH 7.5; 1 mM EDTA and 2 M NaCl were combined and incubated at 95° C. for 2 min, at 65° C. for 10 min, at 37° C. for 10 min and at room temperature for 20 min. To control the performed hybrid formation, 1 μl of the solution was withdrawn and 4 μl of 6×PAGE Loading Buffer and 19 μl of H₂O were combined, separated on 12% PAGE gel (19:1 acrylamide:bisacrylamide) and stained with a SybrGreen (Molecular Probes) diluted 1:1000. In parallel therewith, 1 μl of the biotin anchor and 1 μl of the linker were applied, see FIG. 4. While the single-strand linker runs on a level of 25 bp and the single-strand biotin anchor on a level of 35 bp (biotinylation of this single strand causes the separation to be not completely recise), one can clearly recognize that the anchor linker hybrid runs on a level of 50 bp.

b) Pretreatment of Dynabeads M-280 Streptavidin (according to manufacturer's specifications):

Two hundred μl (2 mg) of Dynabeads M-280 Streptavidin were washed once with twice the volume of a solution of 0.1 M NaOH and 0.05 M NaCl, once with twice the volume of a 0.1 M NaCl solution and resuspended in 375 μl of a solution of 10 mM Tris HCl, pH 7.5, 1 mM EDTA and 2 M HCl.

c) Immobilization of the anchor linker hybrid:

Three hundred seventy-five μl of a solution of 3 mM Tris HCl (pH 7.5), 0.2 mM EDTA and 50 μl of the anchor linker hybrid obtained in a) are added to the solution obtained in b) and incubated at room temperature for 15 min with occasional shaking. Using the provided magnetic support (DYNAL), one washes twice with a solution of 10 mM Tris HCl, pH 7.5, 1 mM EDTA and 2 M NaCl and subsequently resuspends in 200 μl of a solution of 20 mM Tris HCl, pH 7.5, 1 M LiCl, 2 mM EDTA.

d) Isolation of m RNA:

One hundred μg of the whole RNA were heated in 100 μl of H₂O to 65° C. for 2 min and subsequently added to the derivatized beads obtained in c) and freed from solution. The reaction vessel was shaken at room temperature for 5 min, placed in the magnetic support, and subsequently the beads were washed twice with 200 μl of a solution of 10 mM Tris HCl, pH 7.5, 0.15 M LiCl, 1 mM EDTA.

e) Labeling reaction:

The isolated mRNA was washed once with a solution of 3 mM Tris HCl (pH 7.5), 0.2 mM EDTA and two time with 1×RT buffer (GIBSO), Twenty-one μl of H₂O, 8 μl of 5×First Strand Buffer (GIBCO), low C dNTPs (10 mM dATP, 10 mM dGTP, 10 mM dTTP; 4 mM dCTP) (GIBCO), 2 μl of FluoroLink™ CY3/5-dCTP (Amersham Pharmacia), 4 μl of 0.1 M DTT and 1 μl of Rnasin (20-40 u) (PROMEGA) were combined in an Eppendorf reaction vessel and shaken for a short time. This solution was used to re-suspend the isolated mRNA, incubated at 42° C. for 5 min, combined with 1 μl (200 U) of Superscript II (SSII, GIBCO), incubated at 37° C. for 30 min continuous shaking combined with another 1 μl of SSII and shaken at 37° C. for additional 30 min. One added 0.5 μl of RNAseH (GIBCO) and incubated at 37° C. for 20 min.

f) Purification of the labeled probe:

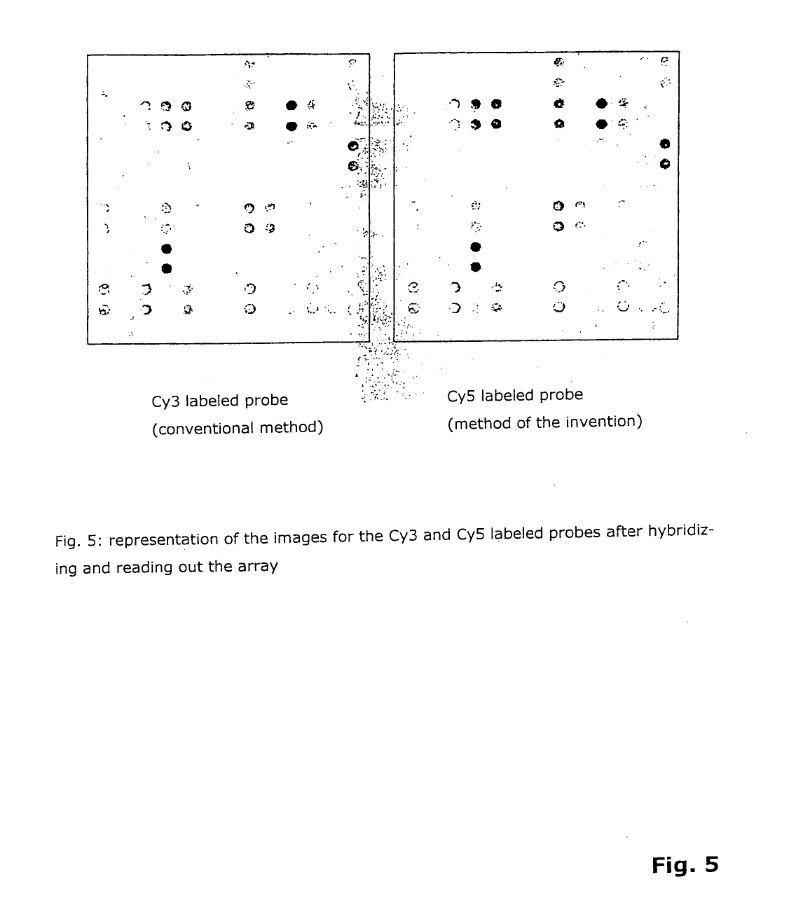

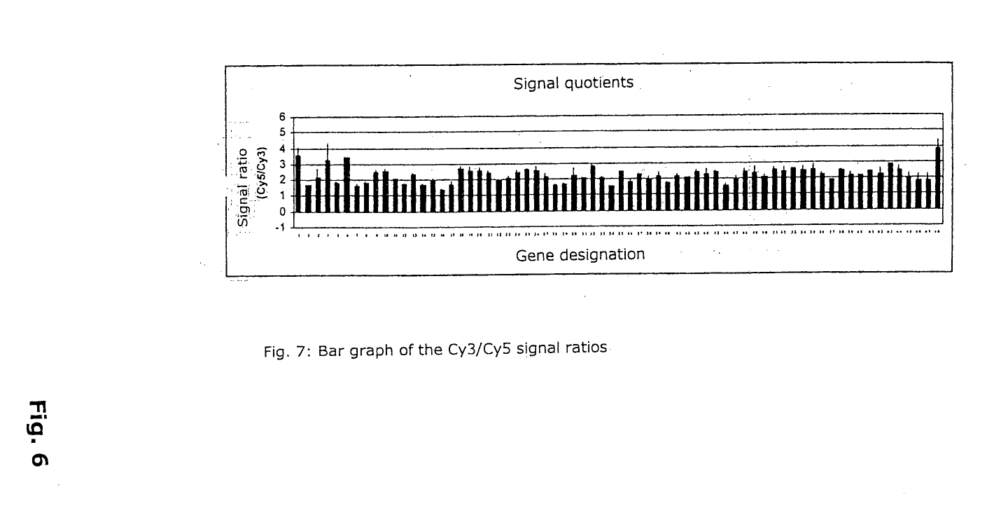

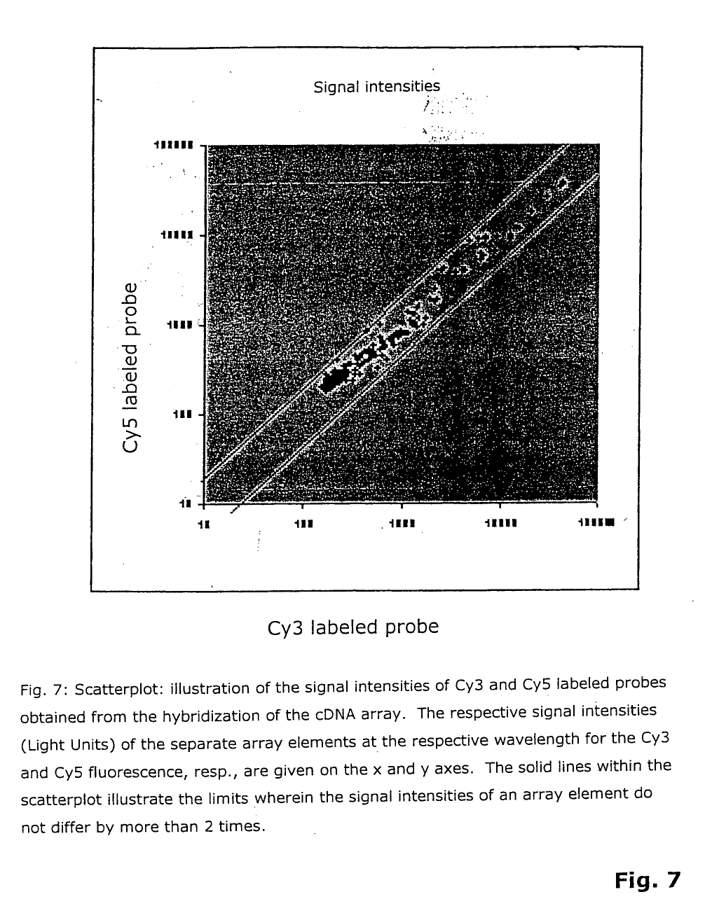

If probes labeled both with Cy3 and Cy5 are to be prepared according to the method of the invention, both labeling reactions may now be combined and processed together. In the sepecial example described here, where the CY5 labeled probe was prepared according to the method of the invention and the CY3 labeled probe was prepared according to the conventional method for comparison, both labeling assays were worked up separated from each other. The probe prepared according to the method of the invention was washed twice with 400 μl of 10 mM Tris HCl, pH 7.5, re-suspended in 10 -82 l of 10 mM Tris-HCl, incubated at 65° C. for 2 min and immediately placed in the magnetic support. The supernatant was transferred into a new Eppendorf reaction vessel. The beads were re-suspended in 10 μl of 10 mM Tris HCl, incubated at 65°0 C. for 2 min, and the supernatant was again transferred into the new Eppendorf reaction vessel. The combined supernatants were combined with the Cy3 probe labeled in the conventional manner, evaporated to 20 μl, provided with 5 μl of a hybridizing solution and applied on a prehybridized cDNA array. The cDNA array was hybridized overnight and subsequently washed with suitable solutions, dried and read out in a laser scanner. The images obtained at different wavelengths (Cy3 and Cy5) are depicted in FIG. 5. Evaluation of the relative signal intensities showed that starting from equal amounts of whole RNA the probe labeled according to the method of the invention on the average provided 2.2 times stronger signals than the conventionally labeled probe, FIG. 6. Further, the evaluation showed that after compensating the absolute signal intensities by a constant normalizing factor calculated from the median value of the signal radio of all signal ratios, none of the array elements hybridized on the cDNA array lead to a signal ration of >±2, FIG. 7. Hence, the variance is not greater than in the comparison of two probes which were labeled in a different way starting from the same RNA by a conventional method using different fluorophores and hybridized on the same array.

1. A nucleic acid complex with an anchor oligonucleotide, an arbitrary nucleic acid or an arbitrary oligonucleotide and a linker oligonucleotide, wherein the linker oligonucleotide is partially hybridized with the anchor oligonucleotide and partially hybridized with the arbitrary nucleic acid or the arbitrary oligonucleotide and the anchor oligonucleotide is immobilized on a support by its 3′-terminus.

2. The nucleic acid complex according to claim 1, wherein a temperature from 35° C. to 85° C., preferably from 45° C. to 65° C. results in an anellation of the anchor oligonucleotide and the linker oligonucleotide.

3. The nucleic acid complex according to any one or more of claims 1 or 2, wherein the hybridizing area of anchor and linker oligonucleotides has a ratio of GC:AT nucleotides from 20:80 to 80:20.

4. The nucleic acid complex according to any one or more of claims 1 to 3, wherein an anellation between the anchor oligonucleotide and the linker oligonucleotide proceeds at a temperature being higher than that of the anellation between the linker oligonucleotide and the arbitrary nucleic acid or the arbitrary oligonucleotide.

5. The nucleic acid complex according to any one or more of claims 1 to 4, wherein the anchor oligonucleotide is immobilized on the support covalently or by an affinity group.

6. A nucleic acid or oligonucleotide hybridization product of an anchor oligonucleotide and a linker oligonucleotide, wherein the linker oligonucleotide is partially hybridized with the anchor nucleotide and the anchor oligonucleotide is partially immobilizable on a support by the 3′-terminus.

7. A method for preparing and separating nucleic acids or oligonucleotides after the synthesis thereof using a nucleic acid complex according to any one or more of claims 1 to 5 or a hybridization product according to claim 6, wherein the following steps are carried out:
attachment of an arbitrary target nucleic acid, the 5′-terminus of which partially hybridizing with the linker oligonucleotide, wherein the linker oligonucleotide is hybridized with the anchor oligonucleotide immobilized on a support;
extension of the linker oligonucleotide by a polymerase and
separation of the extended linker oligonucleotide optionally together with the target nucleic acid.

8. The method according to claim 7, wherein the target nucleic acid is mRNA.

9. The method according to claim 8, wherein the target nucleic acid is degraded by RnaseH or NaOH prior to the separation of the extended linker oligonucleotide.

10. A kit for carrying out the method according to any one or more of claims 7 to 9 comprising
at least one anchor oligonucleotide immobilized on a support by its 3′-terminus and
at least one linker oligonucleotide.


SEQUENCE LISTING (2 sequences)

SEQ ID NO: 1 - DNA, 25 bp, Artificial sequence
  probe
ccccctcgtaagatgagcactctgc

SEQ ID NO: 2 - DNA, 45 bp, Artificial sequence
  probe
agcattctactcgtgagacgccccctttttttttttttttttttt